(19) United States
(12) Patent Application Publication
(10) Pub. No.: US 2005/0020937 A1
(43) Pub. Date: Jan. 27, 2005

(54) Method for detecting pathogenic agents

(75) Inventors: Eddie Reed, Morgantown, WV (US); Barbara Ducatman, Morgantown, WV (US); Timothy Tracy, Shoreview, MN (US); A. Patrick Soisson, Morgantown, WV (US); Pamela Brown, Morgantown, WV (US)

Correspondence Address: PALMER & DODGE, LLP;PAULA CAMPBELL EVANS, 111 HUNTINGTON AVENUE, BOSTON, MA, 02199, US

(73) Assignee: West Virginia University

(21) Appl. No.: 10/625,164

(22) Filed: Jul. 23, 2003

Related U.S. Application Data
(60) Provisional application No. 60/459,180, filed on Mar. 31, 2003.

(51) Int. Cl. A61B 10/00; G01N 33/48

(52) U.S. Cl. 600/562; 436/86; 436/63

(57) ABSTRACT

The invention provides methods and kits for collection of cervical/vaginal specimen. The specimen is collected for the purpose of detecting the presence or absence of etiological agents, e.g., bacterial or viral agents, including but not limited to human papilloma virus (HPV). The endocervical cells is not required for HPV testing or Pap smear. The methods and kits allow the self-sampling without the aid of trained medical personnel.

### PRIORITY

This application claims priority to 60/459,180, filed on Mar. 31, 2003.

### GOVERNMENT SUPPORT

The invention was supported, in whole or in part, by the Centers for Disease Control. The Government has certain rights in the invention.

### BACKGROUND

Cervical cancer remains an important public health problem in the United States and throughout the world. Early detection of precancerous or cancerous conditions attributes significantly to reduction of the incidence of cervical cancer. It is well accepted that conscientious and widespread use of cytologic screening will significantly decrease the incidence and mortality rates of cervical cancer. Cytologic screening typically involves obtaining a sample of cells or tissue from cervix and testing the sample for the presence of cervical carcinoma cells. Currently, the most common method used for this testing is the Papanicolaou (Pap) smear. A pap smear is a microscopic examination of cells scraped from the cervix using sampling apparatus designed for use only by a medical provider. Although Pap smear allows early detection of cervical cancer, Pap smear also requires the medical provider to collect specimen from specific part of cervix. This attributes to at least 4% to up to 20% false negative results due to the incorrect specimen collection. In addition, Pap smear may also involve discomfort, inconvenience, and embarrassment to some patients. It is not affordable to some women, and not accessible in some regions. As a result, many women do not have the Pap smear performed at recommended intervals and cytologic screening to reduce the incidence of cervical cancer may not be fully successfully implemented.

In an attempt to minimize the problems associated with cytologic screening at a medical facility, a number of self-sampling device and method for cervical specimen have been proposed in U.S. Pat. No. 6,155,990, U.S. Pat. No. 6,387,058, U.S. Pat. No. 6,475,165, U.S. Application No. 2002/0032389, U.S. application No. 2002/0120213, and U.S. application No. 2003/0028123. All these self-sampling devices and methods have drawbacks and disadvantages. U.S. Pat. No. 6,155,990, U.S. Pat. No. 6,475,165, U.S. application No. 2002/0032389, and U.S. application No. 2003/0028123 describes using absorbent material such sponge to collect the cervical. Such absorbent material may not take sufficient amount of samples for detection assays, and the absorbent material may also take longer time to release its captured samples and may not release all that it has absorbed. U.S. Pat. No. 6,387,058 and U.S. application No. 2002/0120213 both drawn to the same inventor, describes using a mop-like brush to collect the samples. The collection requires shield being withdrawn when collecting the samples, which renders the device difficult to use and may cause possible contamination from vaginal tract. Furthermore, all these methods require precise location of the devices inside the cervical/vaginal tract. Therefore, there is a need for an efficient and simple-to-use device and method for self-sampling.

Recent progress in scientific studies have revealed that human papillomavirus (HPV) is believed to be the central cause of cervical cancer. HPV infects cervical tissue and the infection is associated with the development of cervical carcinoma. HPV are DNA viruses with a genome size of about 8000 base-pairs. There are more than 100 HPV types based on differences in their DNA sequences. HPV types 16 and 18 referred to as high risk HPVs, are considered carcinogenic. HPV types 31, 33, 35, 39, 45, 52, 56, and 58 have an important role in carcinogenesis. Upon infecting the cells, HPV causes changes in the infected cells' nucleus and cytoplasm.

### SUMMARY OF THE INVENTION

The present invention relates to a device and a self-sampling method for collecting and processing cervical/vaginal specimens. The specimens are collected for the purpose of detecting the presence or absence of etiological agents, e.g., bacterial or viral agents, such as human papilloma virus (HPV), especially “high risk” subtype HPVs that are associated with the development of cancer, including cervical cancer and head and neck cancers.

The method disclosed herein allows the patient to collect the specimens herself, without the aid of trained medical personnel. The method also does not require the presence of endocervical cells in the specimens.

In a preferred embodiment, the present invention provides a method for the accurate detection of HPV from self-collection samples in which the presence of endocervical cells is not required. Surprisingly, it has been discovered that a sample containing only cervical epithelial cells, i.e., containing few or no endocervical cells, is adequate for accurately detecting the presence of HPV and/or for evaluating cervical abnormality.

Specifically, the invention features a method of detecting the presence of HPV in a cervical/vaginal sample, comprising: (a) obtaining a sample of cervical/vaginal specimen; and (b) assaying the sample for the presence of HPV through HPV DNA assays, immunoassays, or any other HPV assays known in the art. The sample may contain few or no endocervical cells. There is no requirement that the sample be collected by medical personnel, rather, the sample may be collected by the patient herself.

The invention also features a device for collecting a sample, wherein the device comprises a brush attached to an end of a support. The brush includes a longitudinal axis, and bristles extending laterally outward from the longitudinal axis. In one embodiment, the device also can include a tubular shield into which the brush can be withdrawn. The shield can be of a length equal to the brush, or it can be longer, or it can be shorter. In a preferred embodiment, the device is substantially as shown in FIGS. 1A and 1B.

The present invention also features a kit for self-sampling of cervical/vaginal specimens. Such kit may include a sample collection device, and optionally, instructions and packaging materials. The kit may also include packaging materials for use in transporting of the sample to a testing sample, e.g., an envelope or rigid shipping container for sending the sample to a laboratory. The kit may also include an envelope for returning the test results to the patient.

In another aspect, the invention also features a home-test kit for detecting the presence of HPV proteins in the sample, wherein the kit includes the collection device, reagents and device for processing and detecting the hrHPV proteins in the sample. Preferable home-test kit can include, in addition to the components included in the home-collection kit above, reagents, antibodies, and enzymes for HPV immunoassays.

In the methods and kits described herein, the HPV may be all types of HPV existing in nature including high risk HPV (hrHPV).

### DESCRIPTION OF THE DRAWINGS

FIG. 1A is a view of self-sampling tool with the specimen-collection element outside the shield.

FIG. 1B is a view of self-sampling tool with the specimen-collection element inside the shield.

### DETAILED DESCRIPTION

The present invention demonstrates that a woman can successfully collect her own cervical/vaginal specimen without the aid of a trained medical provider by using the self-sampling device and method described in the present application, and thereby obtaining a sample that can be accurately assayed for the presence of etiologic agents, such as HPV. In one aspect, the present invention provides a collected specimen that can be further evaluated for the presence of HPV or premalignant conditions of the vigina or cervix, through either HPV assays or Pap smear. In a preferred aspect, the invention provides a method for detecting HPV in vaginal sample that has been self-collected by the patient in which the sample is substantially few of endocervical cells that has been surprisingly discovered that the presence of endocervical cells is not required for HPV testing, and that specimens obtained by the self-collection that are composed mainly of cervical epithelial cells are adequate for detecting the presence of HPV.

The present invention provides a device and a method for a woman to collect her own vagina/cervical specimen for detection of abnormal vagina/cervical conditions through either Pap smear or HPV assays. The present invention is particularly advantageous because (1) the described self-collection method is easy to operate, the time and expense involved in obtaining a sample are reduced, (2) the woman can collect her own cervical/vaginal specimen in a private place, which avoids embarrassment or humiliation and increases patient compliance, (3) the described self-collection method is affordable and accessible to those who can not afford or are unable to obtain physician help, and (4) the specimen collected by the method and device is adaptable for Pap smear and HPV assays.

The present invention further provides methods for detecting the presence of HPV in samples substantially few of endocervical cells. It has been found that endocervical cells are not required for detecting HPV in a cervical/vaginal specimen, and that a sample consisting of cervical epithelial cells is adequate for detecting HPV and/or for evaluating cervical abnormality.

The present invention also is directed to a novel device and a self-sampling method to obtain a sample of cervical/vaginal specimen for HPV assays and/or Pap smear. Preferred embodiments of the invention are described below in conjunction with the drawings, and are not to be construed as a limitation of the scope of the present invention.

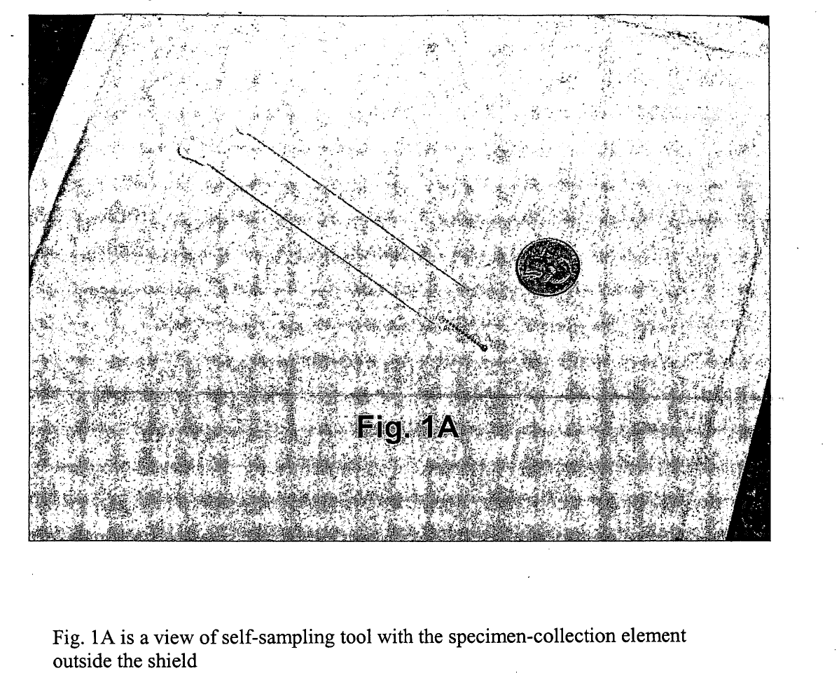

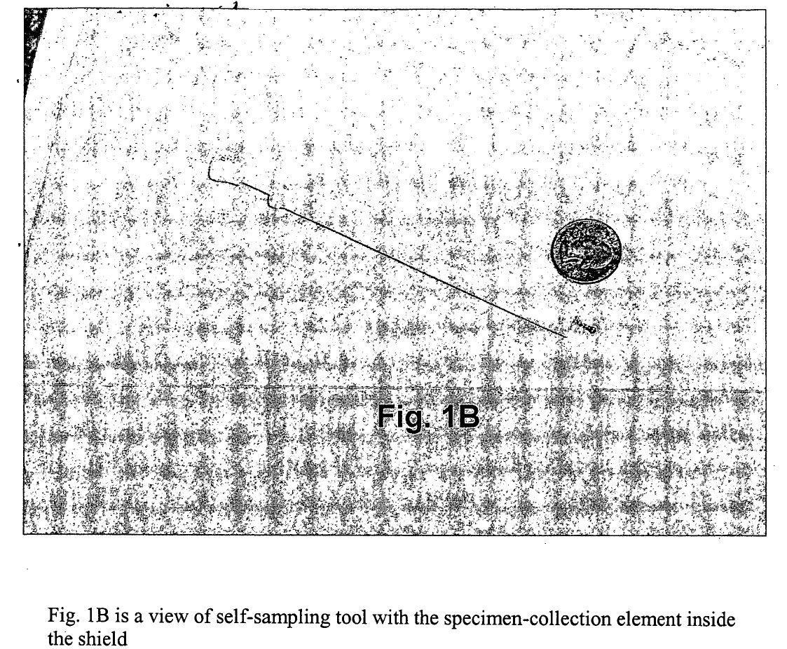

As shown in FIGS. 1A and 1B, the device comprises a brush attached to an inner tube, and an outer tube that serves as shield to the brush and the inner tube. The brush includes a longitudinal axis that runs through the inner tube and bristles that extend laterally outward from the longitudinal axis. The brush and the inner tube as a whole are called the collection element. The inner and outer tubes are preferably cylindrical in shape, and are approximately equal length. The collection element can be removed from the shield for sample analysis. Alternatively, the bristles can have short axis that is mounted to the base of the inner tube, and the bristles can be removed from the base of inner tube.

The total length of the collection element is approximately between 10 cm to 16 cm, preferably 15 cm in length. The length of the inner and outer tubes is approximately between 8 cm to 14 cm and between 7 cm to 13 respectively, preferably 13 cm and 11.5 cm respectively. The diameter of the inner and outer tubes is approximately between 0.85 cm to 1.5 cm and between 0.9 cm to 2.0 cm respectively, preferably 0.9 cm and 1.3 cm respectively. The bristles are approximately between 1.0 to 3.0 cm, preferably 2 cm in length, and the diameter of the bristles is approximately the same as that of the outer tube (0.9-2.0 cm) with smaller diameters at the tip of the bristles. The bristles can be made of any appropriate material known to one skilled in the art. The bristles are preferably made of a flexible plastic material such as polyethylene, polyurethane, polyvinyl chloride, polysiloxanes or nylon, etc.

In the self-sampling method described herein, one preferred embodiment comprises inserting the collection device into the vagina, protruding the collection element out to have the bristles contact with the cervical/vaginal tissues, rotating the inner tube of the collection element, withdrawing the collection element back into the shield, and taking the whole collection device out of the body. The bristles containing the vaginal sample is then immersed into a liquid collection medium.

In a preferred embodiment, the collection tool may incorporate a temporary “lock” or “stop” means to maintain the bristle portion of the collection element inside the shield during insertion into the vagina. The collection element is then inserted into the vagina, and the bristle portion of the collection element is extended so that the bristles emerge from the shield, allowing cervical/vaginal fluid to deposit on the bristles. After cervical/vaginal fluid has deposited on the bristles, the bristles are withdrawn back into the shield, and the entire collection device is removed from the cervix/vagina.

The specimen collected with the described method can then be transferred into a container for further analysis. The container should also contain a liquid medium for preserving the cells that can be used for making a liquid based Pap test or assays for infectious agents including but not limited to HPV and chlamydia. To get most of the vaginal sample transferred from the bristles to the medium, the bristles need to be swirled through the collection medium at least 10 times. Generally, the liquid collection medium is alcohol-based preservative medium which are commercially available by companies such as Cycletech, DIGENE, etc.

One preferred embodiment provides that the cervical/vaginal specimen collected by the self-sampling method of the present invention contains mainly cervical epithelial cells, and that endocervical cells which are majority cells found in a provider-collected specimen, are not required for detecting HPV presence in the cervix, because cervical epithelial cells are shown to be equally adequate for HPV assays. See working examples below. The lack of requirement for endocervical cells has several advantages. It does not require precise location of the collection element inside the cervical/vaginal tract, makes the procedure easy to operate by the patient having no training in collecting cervical/vaginal specimen. It further reduces discomfort, pain or embarrassment related to the collection of the specimen, and encourages more women to participate in the examination. Dzuba et al. (2002, J. Womens Health Gend. Based Med. 11(3):265-275) measured acceptance by women of self-sampling methods vs. a standard Pap smear, and found that women consistently experiences less pain, discomfort and embarrassment with self-sampling methods.

Once a sample of vaginal vault fluid has been obtained, it can be tested for the presence of etiological agents, e.g., HPV. The presence of HPV DNA has been found to be associated with high-grade cervical squamous intraepithelial lesions and invasive cervical canver, and HPV testing has been found to be equal or superior to a standard Pap smear in sensitivity (Wright et al., 2000, JAMA 283(1):81-86).

One way of detecting the presence of HPV is to use Hybrid Capture II technology developed by Digene, which is incorporated wholly by reference herein. The hybrid capture II assay is a second-generation DNA probe test based on signal amplification, which uses a chemiluminescent readout to indicate the presence of one or more carcinogenic HPV types as a group (types 16, 18, 31, 33, 35, 39, 45, 51, 52, 56, 58, 59, and 68).

Another way of detecting the presence of HPV is to use real-time polymerase chain reaction (real-time PCR) techniques as described in Mackay et al., 2002, Nucleic Acid Research 30(6):1292-1305, and Hart et al., 2001, JCM 39:3204-3212, which are wholly incorporated by reference herein.

Another way of detecting the presence of HPV is to use the immunological methods, such as the one described in WO 0177142, which is incorporated by reference herein. Briefly, the method comprises reacting a sample of body fluid or tissue likely to contain antibodies to specific regions of HPV proteins created by using one or more peptides derived from certain HPV proteins. The complex formed by the binding of antibodies to the peptides are detected with conventional immunoassays. This immunological methods can be performed in different manners well known in the art, one of which is ELISA.

There have been several studies comparing self-collected samples with that collected by a medical provider. Nobbenhuis et al. (2002, J. Clin. Pathol. 55(6):435-439) studied self- versus physician-collected samples in an effort to improve rates of participation of women who avoided testing altogether. They found that the concordance rates of HPV detection in doctor- vs. patient-collected samples was 93% for cervical smears, and 78% for cervicovaginal lavage. The authors concluded that self-sampling for HPV DNA is a feasible alternative in women who decline to participate in cervical screening programs. Like Nobbenhuis et al., they concluded that self-sampling would be beneficial for increasing participation by some women who previously refused to take part in screening programs. Harper et al. (2002, Am. J. Obstet. Gynecol. 186(3):365-373) also studied detection of HPV in samples self-collected with swabs and tampons, and concluded that two sequential swabs were better able to detect HPV.

Another preferred embodiment provides that the device can be packaged into a kit for practicing the methods described herein. Such a kit would contain, at the very least, the collection device. Preferably, the device is wrapped to prevent contamination of the sample compartment and prevent the risk of infection to the patient. The compartment in the device within which the sample is collected can be sealed to prevent loss of the sample. The compartment can also be made to be detachable from the overall collection device.

In situations where the sample is collected by the patient in a medical facility, the kit can consist of the collection device, preferably individually wrapped. The kit can also include the device and accessories for cleaning the vaginal area before insertion of the device. Such a kit can also include means for sealing the compartment containing the fluid, to prevent loss of the sample before testing. The kit can also include a secondary container in which the collection device is placed before turning the sample over to medical personnel. Such a container, or the collection device itself, can include means for labeling the device/sample with information identifying the patient as the source of the sample.

The sample can also be sent to a testing facility. In such a situation, the kit can include items such as packaging and shipping materials, such as a shipping container, packaging material to prevent damage to or loss of the sample, and materials for returning the results of the analysis.

The invention also includes kits for practicing the method without involvement of medical personnel, i.e., a “home-use” kit. Such a kit would include the collection device, means for sealing the compartment within which the sample is collected (or alternatively, a second sample container for transfer of the sample). The sample collection compartment can be detachable from the overall collection device. The kit can also include a secondary container in which the collection device is placed before turning the sample over to medical personnel. The container, or the collection device itself, can include means for labeling the device/sample with information identifying the patient as the source of the sample. The kit can also include accessories for cleaning the vaginal area before insertion of the device. The collection device, and various other components of the kit, can be individually wrapped.

A kit intended for home use would also include shipping materials for sending the sample to a testing facility, such as a shipping container, wrapping materials, etc. The kit can also contain materials for return of the results of the analysis to the patient.

The invention can also include a home-test kit for detecting the presence of HPV proteins using HPV-specific antibodies. Such a kit can include the collection device, reagents and device for processing and detecting the hrHPV proteins in the sample. Preferable home-test kit can include, in addition to the components included in the home-collection kit above, reagents, antibodies, and enzymes for HPV immunoassays. For example, in one embodiment, the kit can include a first antibody that specifically binds to HPV proteins in the sample, and a second antibody that specifically binds to the first antibody. The second antibody can be immobilized to a solid support. Upon binding to the first antibody/HPV protein complex, the second antibody can go through reactions and change color. The color indicates the presence of HPV protein in the sample. Alternatively, the kit can include only the first antibody that specifically binds to the HPV proteins in the sample. The first antibody is preferably immobilized to a support, and the binding of the antibody to the HPV proteins will cause reactions and change of color. In another preferred embodiment, the antibody is monoclonal antibody.

Any of the kits described herein can also include instructions for use, and advice in the event of a positive result.

### EXAMPLES

### Example 1

### Collection of Samples and Number of Tests

The study was part of the BCCSP program (Breast and Cervical Cancer Screening Program), which was sponsored by the CDC (Centers for Disease Control, Atlanta, Ga., USA).

Ninety patients were included, with an age range of 26 to 63 years, and a median of 45 years. The age range by decade of life is show below.

TABLE 1
Age distribution of women participating the study

| Age range by decade of life | Percent of total |
|---|---|
| 20-29 years of age | 4.8% |
| 30-39 years of age | 29.8% |
| 40-49 years of age | 34.5% |
| 50-59 years of age | 23.8% |
| ≧60 | 8.1% |

Two samples were collected from each of ninety patients. For each patient, one sample was collected by a health care provider, while the other sample was collected by the patient herself. Two of the ninety samples collected by the health care provider were of insufficient volume to be confident in the test results (2.22%). Five of the ninety patient-collected samples were of insufficient volume to be confident in the test results (5.55%). For those five samples that were of insufficient volume, two were matched with positive HPV results from the “provider collected” specimens; two were matched with negative HPV results from the “provider collected” specimens; and, in one, both specimens (provider collected and self collected) were of insufficient volume.

TABLE 2
Comparison of Provider collected sample to Self-collected sample

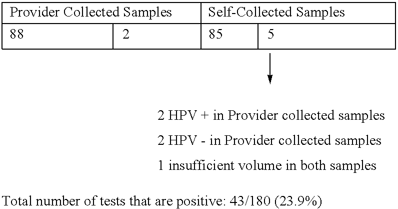

18/90 Provider collected specimens were HPV positive
   Endocervical cells were seen in 79/90 (87.8%) specimens
25/90 Self collected specimens were HPV positive
   Endocervical cells were seen in 9/90 (10%) specimens
Of eighteen (18) “provider collected” HPV positive specimens:
   Seventeen (17) specimens had endocervical cells seen at Pap.
   One (1) specimen was negative for endocervical cells at Pap.
Of twenty-five “self collected” HPV positive specimens:
   One (1) specimen had endocervical cells seen at Pap.
   Twenty-four (24) specimens were negative for endocervical cells at Pap.

Thirteen (13) individuals were HPV positive in both specimens. In twelve cases, endocervical cells were seen in the “provider collected” specimen; but no endocervical cells were seen in the “self collected” specimen from the same individual. In one individual, endocervical cells were seen in both specimens.

Surprisingly, the presence or absence of endocervical cells appeared to have no appreciable affect on determining whether or not the sample was HPV positive. Endocervical cells were rarely seen in those specimens that were “self collected”. The presence of endocervical cells is therefore not necessary in determining HPV positivity of a sample.

### Example 2

### HPV Positive Individuals

| | |
|---|---|
| Total number of individuals HPV positive | 30/90 (33.3%) |
| (either test): | |
| Number positive on both tests: | 13/90 individuals (14.4%) |
| Number positive on “provider collected” | 5/90 individuals (5.5%) |
| only: | |
| Number positive on “self collected” only: | 12/90 (13.3%) |
| Age range of HPV positive individuals: | 29 to 60 |

The “provider collected” specimens picked up 18 of the 30 (60%) of HPV positive individuals; whereas “self collected” specimens, picked up 25 of the 30 (83.3%) of HPV positive individuals.

### Example 3

### Cellular Abnormalities/Changes Seen

Number of HPV positive individuals, no cellular abnormalities: 25/30
Number of HPV positive individuals, cellular abnormalities seen: 5/30

Number of individuals with cellular changes/abnormalities (−) for HPV: 18/60

| | |
|---|---|
| Reactive epithelial cells = | 5/60 |
| Inflammation = | 12/60 |
| ASCUS* = | 1/60 |

*atypical squamous cells of undetermined significance

Most of the HPV positive cases showed no cellular abnormalities at Pap. Most of the cases with cellular changes at Pap were negative for HPV. These changes were primarily inflammation and reactive epithelial cells. The one case where LGSIL (low-grade squamous intraepithelial lesion) was seen at Pap was HPV positive.

### Example 4

### Age May Influence Rate of Positive hrHPV Testing and the Viral Load

Women are screened for hrHPV positively, using ThinPrep and Hybrid Capture II (HCII) methodology, in the CDC-sponsored BCCSP Program in the State of West Virginia. The first 163 individuals screened are included in this analysis. Age range was 25 to 63 yrs; median 44. HCII assays were performed, blinded to clinical information and blinded to cytological assessment. All patients had two specimens assayed; 1 self collected specimen, and 1 provider collected specimen. 326 HCII tests were performed. Patient data were stratified by <50 years old versus ≧50 years old. 26/102 individuals <50 were hrHPV positive (25.5%); 6/46 patients ≧50 were hrHPV positive (13.0%); and 3/15 patients age not known were positive (20%). Rate of positivity was higher in younger patients, but did not reach statistical significance; p2=0.0442. The hrHPV positive rate in younger patients is consistent with previously published reports; whereas the 13% rate in older patients is 3-4 fold higher than predicted. The level of HCII positive signal was assessed as well, and the data were stratified by age. The level of signal is presumed to reflect the amount of hrHPV in the specimen; i.e., viral load. Younger patients had hrHPV levels that were consistently higher than those seen in older patients. Median levels differed by a factor of six, and mean levels differed by more than a log, p2=0.0097. We conclude that hrHPV rates are higher in younger patients in our population; and that among hrHPV positive individuals, the viral load might be higher in younger patients.

### Example 5

### Screening for hrHPV in a Stable Appalachian Population and its Relation To Age

Women are screened for hrHPV positivity, using ThinPrep and Hybrid Capture II methodology, in the CDC-sponsored BCCSP Program in the State of West Virginia. The first 163 individuals screened are included in this analysis. Age range was 25 to 63 years; median 44. HCII assays were performed, blinded to clinical information and blinded to cytological assessment. All patients had two specimens assayed; 1 self collected specimen, and 1 provider collected specimen. 326 HCII tests were performed. 33 patients had one or both specimens test positive for hrHPV; age range was 29 to 61, median 40. Age distribution of patients was: 25-29 yrs, 10 patients (6.1%); 30-39 yrs, 40 patients (24.5%); 40-49 yrs, 52 patients (31.9%); 50-59 yrs, 38 patients (23.3%); 60-63 yrs, 8 patients (4.9%); and age not declared, 15 patients (9.2%). hrHPV positivity within these age groups was: 25-29 yrs, 1/10; 30-39 yrs, 15/40; 40-49 yrs, 10/52; 50-59 yrs, 5/38; 60-63 yrs, 2/8; and age not declared, 3/15. Therefore, percent positivity within each designated age group was: 25-29 yrs, 10%; 30-39 yrs, 37.5%; 40-49 yrs, 19.2%; 40-49 yrs, 13.2%; 60-63 yrs, 25%; and age not declared, 20%. Although younger patients tended to be positive in a higher percentage of cases, our rate of hrHPV positivity was in the range of 15-20% in individuals >40 years of age. This is 3-4 fold higher than what would be predicted based on previously published literature in other populations. There was no statistically significant difference between rates of positivity between groups in their 40's, 50's, or 60's

### Example 6

### Screening for hrHPV in a Stable Appalachian Population and its Relation to the Method of Sample Collection

Women are screened for hrHPV positivity, using ThinPrep and Hybrid Capture II methodology, in the CDC-sponsored BCCSP Program in the State of West Virginia. The first 163 individuals screened are included in this analysis. Age range was 25 to 63 years; median 44. HCII assays were performed, blinded to clinical information and blinded to cytological assessment. All patients had two specimens assayed; 1 self collected specimen, and 1 provider collected specimen. 326 HCII tests were performed. Whereas 36/163 (22.1%) individuals were positive on at least one test, only 14/163 (8.6%) were positive on both tests. 28/163 specimens were hrHPV positive in self collected specimens (17.2%), although only 23/163 specimens showed endocervical cells on cytologic examination. 22/163 specimens were hrHPV positive in provider collected specimens (13.5%), and 133/163 specimens showed endocervical cells on cytology. This suggests that the presence of endocervical cells was not necessary to detect infection with hrHPV. In these 163 individuals, LGSIL (low-grade squamous intraepithelial lesion) and ASCUS (atypical squamous cells of undetermined significance) were observed, but no cases of HGSIL (high grade squamous intraepithelial lesions), CIN (cervical intraepithelial neoplasia), nor invasive cancer were observed. Data from HCII testing were analyzed to assess whether the level of positivity (presumed to reflect viral load) was influenced by whether the sample was self collected or provider collected. Medians and means from positive samples that were self collected or provider collected were in the same range, with p2=0.2493. We conclude that the presence of endocervical cells is not necessary for the assessment of hrHPV; which implies that vaginal epithelial cells are a good source for this assessment, as are cells from the cervix.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### Example 7

### Adequacy of Self-Collected Samples for Human Papillomavirus (HPV) Testing and Detection of Cervical Cancer

From July 2002 to January 2003, 274 women in rural Appalachian were enrolled in a study for detection of hrHPV and cervical cancer, and the adequacy of self-collected versus physician/provider collected samples for HPV testing and cytologic diagnosis was evaluated. After enrollment, the women were given written instructions on how to obtain the vaginal specimens. After self-collection, each woman underwent a pelvic examination, which included collecting vaginal samples by trained providers. At the end of the study, each woman completed a self-administered questionnaire, which included questions about sociodemographic characteristics and sexual behavior. Each woman was also asked to grade/rank the acceptability of the self-collected method. HPV in the specimens was detected using ThinPrep and Hybrid Capture II (HCII) methodology.

The results showed that the average age of the 274 women was 43.3 years, and that younger women are more likely to be hrHPV positive. Among the 274 women, the average age of HPV positive women was 40.5, and the average age of HPV negative women was 44.0. Among the 274 women, 54 women were found to be HPV positive (20%). All women had two specimens assayed: one self-collected specimen (SC), and one physician or provider collected specimen (PC). The HCII results showed that 12/274 (4%) of the women were found HPV positive on PC specimens; 21/274 (8%) of the women were found HPV positive on SC specimens; and 21/274 (8%) of the women were found positive on both specimens.

As to the distribution of endocervical components by specimen type, PC specimens are more likely than SC specimens to have endocervical components. The prevalence of the endocervical components was 75% (202/268 women) in PC specimens and 13% (35/268 women) in SC specimens. The prevalence of hrHPV was 12% (33/268 women) in PC specimens and 16% (42/268 women) in SC specimens. Among the 42 hrHPV positive women with SC specimens, only one was found to contain endocervical components, whereas among the 33 hrHPV positive women with PC specimens, 25 women were found to contain endocervical components. The results are shown in Tables 3 and 4.

TABLE 3
hrHPV vs. Endocervical component in PC specimens

| | hrHPV negative | hrHPV positive | Total |
|---|---|---|---|
| Endocervical | 58 | 8 | 66 |
| component negative | | | |
| Endocervical | 177 | 25 | 202 |
| component positive | | | |
| Total | 235 | 33 | 268 |

TABLE 4
hrHPV vs. Endocervical component in SC specimens

| | hrHPV negative | hrHPV positive | Total |
|---|---|---|---|
| Endocervical | 192 (85%) | 41 (98%) | 233 |
| component negative | | | |
| Endocervical | 34 (15%) | 1 (2%) | 35 |
| component positive | | | |
| Total | 226 | 42 | 268 |

The endocervical components were also evaluated for HPV testing. The results showed, surprisingly, that the presence of endocervical cells was not necessary to obtain accurate results in HPV testing (See Table 5). For example, among the specimens found negative for an endocervical component, 18% (11/60) of the women were hrHPV positive. Moreover, in SC specimens found positive for an endocervical component, 17% (1/6) of the women were hrHPV positive. In PC specimens found positive for an endocervical component, 23% (40/173) of the women were hrHPV positive.

TABLE 5
Presence of endocervical component vs. hrHPV results

| | | | | |
|---|---|---|---|---|
| hrHPV negative | SC | PC | Both | Total |

| | | | | | |
|---|---|---|---|---|---|
| Endocervical | 49 | 4 | 3 | 4 | 60 |
| component | | | | | |
| negative | | | | | |
| SC | 5 | 0 | 1 | 0 | 6 |
| PC | 133 | 17 | 7 | 16 | 173 |
| Both | 27 | 0 | 1 | 1 | 29 |
| Total | 214 | 21 | 12 | 21 | 268 |

The PC and SC specimens were also examined by a Pap smear test. The results showed that 15/274 (5%) of the women were found abnormal in both SC and PC specimens. Accordingly, the self-collection method is also acceptable for Pap test. See Table 6.

TABLE 6
Comparison of Pap test results in PC vs. SC specimens

| | Negative (PC | Unsatisfactory | Positive (PC | |
|---|---|---|---|---|
| | specimens) | (PC specimens) | specimens) | Total |
| Negative (SC | 248 | 4 | 4 | 256 |
| specimens) | | | | |
| Unsatisfactory | 1 | 2 | 0 | 3 |
| (SC specimens) | | | | |
| Positive (SC | 4 | 0 | 11 | 15 |
| specimens) | | | | |
| Total | 253 | 6 | 15 | 274 |

The Pap test and the hrHPV test were compared in both PC and SC specimens. The results showed that total 20% of the women (54/268) were found hrHPV positive, whereas total 7% of the women (19/268) were found to have abnormal Pap test (See Table 7.). The results indicate that the HPV test is a more sensitive methodology in detecting early stage of cervical/vaginal abnormality.

TABLE 7
Correlation of abnormal Pap test with presence of hrHPV

| | hrHPV | +hrHPV in | +hrHPV | +hrHPV | |
|---|---|---|---|---|---|
| | negative | SC | in PC | in Both | Total |
| Negative | 205 | 21 | 9 | 14 | 249 |
| Pap test | 96% | 100% | 75% | 67% | |
| +Pap test | 3 | 0 | 0 | 1 | 4 |
| in SC | 1% | | | 5% | |
| +Pap test | 2 | 0 | 1 | 1 | 4 |
| in PC | 1% | | 8% | 5% | |
| +Pap test | 4 | 0 | 2 | 5 | 11 |
| in Both | 2% | | 17% | 24% | |
| Total | 214 | 21 | 12 | 21 | 268 |

1. A method for detecting the presence of human papilloma virus (HPV) in a vaginal specimen, said method comprising:
(a) obtaining the vaginal specimen wherein the specimen contains few or no endocervical cells;
(b) extracting DNA from the specimen; and
(c) detecting the presence of HPV DNA in the specimen.

2. A method for detecting the presence of human papilloma virus (HPV) in a vaginal specimen, said method comprising:
(a) obtaining the vaginal specimen wherein the specimen contains few or no endocervical cells;
(b) contacting the specimen with a polypeptide that binds to a HPV antibody; and
(c) detecting the presence of the HPV antibody in the specimen.

3. A method for detecting the presence of human papilloma virus (HPV) in a vaginal specimen, said method comprising:
(a) obtaining the vaginal specimen wherein the specimen contains few or no endocervical cells;
(b) contacting the specimen with a polypeptide that binds to a HPV protein; and
(c) detecting the presence of the HPV protein in the specimen.

4. A method for detecting the presence of human papilloma virus (HPV) in a vaginal specimen of a patient, the method comprising:
(a) obtaining the vaginal specimen by self-collection by the patient, wherein the specimen contains few or no endocervical cells; and
(b) assaying the specimen for the presence of HPV.

5. The method of claims 1-4, wherein the HPV is high risk HPV (hrHPV).

6. A kit for detecting the presence of human papilloma virus (HPV) in a specimen, comprising a specimen collection device for obtaining a sample containing few or no endocervical cells, and optionally instructions for use and packaging materials.

7. The kit of claim 5, wherein the HPV is high risk HPV (hrHPV).
